(19) United States
(12) Patent Application Publication
(10) Pub. No.: US 2012/0150316 A1
(43) Pub. Date: Jun. 14, 2012

(54) ESOPHAGEAL FLOW CONTROLLER

(76) Inventor: Jose da Conceiçao Carvalho, Goiania (BR)

(21) Appl. No.: 13/133,248

(22) PCT Filed: Aug. 31, 2009
(86) PCT No.: PCT/BR09/00346

§ 371 Date: Jun. 7, 2011

(51) Int. Cl. A61F 2/04; C08L 7/02; B29C 65/52; B29C 39/22; B29C 39/02

(52) U.S. Cl. 623/23.68; 264/39; 264/305; 156/242; 264/219; 524/575.5

(57) ABSTRACT

The object of the present patent is characterized by being a brand-new device which was developed to control the esophageal flow; it is made of biosynthetic material based on the natural latex extracted from the rubber tree Hevea brasiliensis, to be applied in the esophagus in order to control or limit the speed and amount of food intake and, thus, to provide weight loss. It preserves all digestive system functions, decreases food intake with the reduction of the esophagus lumen and, in turn, decreases the organ emptying, thus controlling the speed and amount of food intake.

The “ESOPHAGEAL FLOW CONTROLLER”, to be used together with a technique for controlling and treating obesity, to control the volume and the speed of the food intake, stimulate a higher individual's chewing rate, thus decreasing the amount of food intake; being adjustable to the esophagus lumen and to control the esophageal flow and the ESOPHAGEAL FLOW CONTROLLER being provided with a flexible and inflatable external part of a conductor duct with a fixed diameter, with a valve to let air in and with a flexible wire that enables its setting and removal, made of biosynthetic material, specifically but not limited to the natural latex extracted from the rubber tree Hevea brasiliensis and be constituted by the whole EFC set (1), external tube (2), internal tube (3), surgical wire (4), valve (5), scalp (6), transversal grooves (7), suction cups (8) and a radiographic indicator (9).

Eating is a part of human behavior that depend on motivation. The amount of food intake is influenced by non-biological factors which include social circumstances, cultural customs, cost, convenience, and period of the day. Nevertheless, physical and biological factors such as sight, smell, taste and, especially, the basal metabolical rate and the size of its spares of energy also regulate the appetite (KAULING 2007).

However, due to the uncontrollable eating act, an epidemic which is the pathology called obesity can be noticed in the current society. Obesity has become a serious public health problem, due to its high prevalence, the difficulty in controlling it and the high rate of recurrence (PETRIBU et al. 2006).

Obesity must be recognized as a serious disease responsible for premature deaths and for the mortality of millions of people, besides being responsible for serious individual problems concerning the psychosocial status and quality of life.

Obesity causes are not thoroughly understood and defined in the literature yet. It is known to involve genetic, physiological, metabolic and appetite cerebral regulation factors as well as environmental and psychosocial conditions besides cultural factors (DOHERTY 1999, SUDO et al. 2007, HALPERN et al. 2004).

The. World Health Organization (WHO) classifies obesity as a problem of worldwide public health and as a chronic disorder and it is already listed in the International Classification of Diseases (ICD-10) as E-66. Its growing rate status and high prevalence demand more objective deeds from the authorities. Since 1980, an increased prevalence of over 75% has been noticed; being the children and youngsters the most affected population. Nowadays, it is estimated that there are around 300 million obese people in the world, and this number tends to double by 2025 unless efficient measures are taken. In a recent poll, the Brazilian Association of Endocrinology and Metabolism (SBEM-BAEM) shows that obesity has increased by 240% in the past 20 years in Brazil.

Obesity is responsible for a poor quality of life. It influences with the individual's physical and mental well-being and affects people regardless of race, sex, age or ethnic. Science has already discovered and clarified some conflicting theories and treatment techniques, but this is still a field with many defined-to-be researches as there are many pieces of unexplored and conflicting information in literature, as can be noticed.

An individual is regarded as obese when their Body Mass Index (BMI) is above the value considered to be normal or overweight. In order to calculate a person's BMI, one must divide their weight in kilograms by their square height in meters (Kg/m²). In general, an index which is lower than twenty-five is regarded to be normal whereas one between twenty-five and thirty is considered to be overweight.

Thus, it can be stated that obesity is one of the major problems in public health and it has been reaching epidemic proportions both in developed countries and in those developing. It is a pathology determined by the association of several factors and it is this multiple cause that makes its treatment even more difficult.

There are several ways to try to treat obesity, such as diets, medications, surgical interventions and surgical treatments. Regarding the treatments through diets and medications, the efficiency and success of many of these proposals are known to be not completely satisfactory as these treatments are unable to offer a definitive loss of weight. This is due to several factors, such as: the obese person's indiscipline, localities where they live with others, behavioral factors, slow result that drives the patient impatient, besides other factors (DAMIANI et al. 2000).

Taking the inexistence of really efficient alternatives into account, the present ESOPHAGEAL FLOW CONTROLLER—EFC—has been developed.

The present invention describes a new device to be used together with a technique for controlling and treating obesity, classified as a restrictive technique which has the same usage principle as the one applied in the adjustable gastric band and in the intragastric balloon introduced by several companies.

The ESOPHAGEAL FLOW CONTROLLER is adjustable to the esophagus lumen and it is provided with a flexible and inflatable external part of a conductor duct with a fixed diameter, with a valve to let the air in and with a flexible wire that enables its setting and removal. Such characteristics aim to control the volume and the speed of food intake, stimulate a higher individual's chewing rate, thus decreasing the amount of food intake and, consequently, obtaining effective loss of weight.

Remarks about the Known State of the Art:

The eating act is part of the human behavior that depends upon motivation. The food intake is influenced by non-biological factors which include social circumstances, cultural customs, cost, convenience and the period of the day (KAULING 2007). However, physical and biological factors such as sight, smell, taste and, particularly, the body's basic metabolic rate and the size of its spares of energy regulate the appetite. As in our today's society there is an epidemic due to the uncontrollable eating act, the pathology so-called obesity arises, which is a current serious problem to the public health considering its high prevalence, the difficulty for it to be controlled and the high rate of recurrence (PETRIBU 2006).

Many treatment proposals for obesity have been developed in the past years. Among them, there are therapeutic, medical and surgical techniques. The two last ones are generally based on the gastrointestinal physiology alteration, which brings about significant morbidity and mortality rates related to those procedures as well as it may lead to a deficit of vitamins and nutrients, besides hormonal alterations.

Several treatment techniques for controlling obesity are described in literature (MARTINS 2005; BROLIN 2002; MARTIN 1995; TONETO et al. 2004; ALMEIDA 2006, MIGUEL et al. 1994), and all of them have advantages and disadvantages. In MARTINS 2005 e BUCHWALD et al. 2004, it is stated that about 65% of the techniques are based on the gastric bypass or gastric surgery with “Y” derivation. This sort of procedure, besides performing a mechanical factor of restriction and bad absorption, may also lead to a decrease of exogenous hormones, deficiency of vitamin B1 and may cause the syndrome called Wernicke Encephalopathy, which may result in sight problems (SINGH et al. 2007). There are other less accounted problems that are also caused by obesity, such as: psychological and social disorders that are derived from physical limitation, low self-esteem and difficulty in being inserted in the scenery advertised by the media. Therefore, there is a real need of having other methods created for therapeutic control of obesity, as the ones that exist today cannot yet reach their goals without bringing damage or high costs.

In documents WO01/24742; U.S. Pat. No. 4,592,339A, WO03/020183A1, BRP10412605-0, BRPI0402538-5 and BRPI0602795-4, the variations of the ways of placing the gastric band are described as follows: a band placed around the stomach with a mechanical constriction before a strangulation; settling of a chamber that keeps the band facing the stomach that, when filled with liquid, it controls the stomach expansion; the band is involved in a viscoelastic material for protecting the stomach; development of a double chamber to control the food. Regarding the intragastric balloon in the documents BRPI10108967-6, BRPIO215432-3, BRP10408867-0, BRPI0413956-9, BRPI0415983-7, BRPI0507948-9, BRPI0507926-8, the place to be inserted inside the stomach to reduce its volume; the construction alterations and principles are described. However, all the introduced methods have a common thread, that is, they all attempt to control the food intake volume.

Taking into account that obesity is a clinical, chronic and a multi-causal etiologic disorder, its treatment, despite the intervention that is carried out, must be long-term and reach all the different areas that are involved. Nowadays, the following obesity treatment methods can be found in literature:
Diets;
Non-pharmacological treatment;
Pharmacological treatment;
Surgeries.

All the methods mentioned above have something in common: decreasing the food intake—as, in order to lose weight, it is necessary to bring about an energetic deficit, by limiting amount of nutrient absorption. Each with its own way, technique and planning, they all attempt to reach that very goal—with related advantages and disadvantages.

Among the available techniques for treating obesity, the ones that mostly reach their goal of decreasing the food intake are the surgical ones, due to the mechanical and physical restrictions they subject the patient to. There are three basic types of surgical treatments:
Disabsorptive techniques: Scopinaro surgery and duodenal switch;
Restrictive techniques: the intragastric balloon, vertical gastric surgery with a band (Mason surgery); adjustable gastric band;
Blended techniques: Fobi-Capella surgery.

Nevertheless, among the aforesaid treatments classified as being surgical, the ones that limit the gastric volume without great surgical intervention are the adjustable gastric band—made through video laparoscopy, and the intragastric balloon—made through endoscope. Both promote the reduction of the gastric volume (stomach) by means of a mechanical system, by reducing the amount of food to be received by the stomach.

The adjustable Gastric Band is consisted of a silicone prosthesis that has an inflatable balloon inside it. It is placed around the highest part of the stomach forming a ring that tightens it, giving it the shape of a sand clock. When the balloon is inflated or deflated, it tightens the stomach more or less so that to control the emptying of the food from the higher part to the lower part of the organ. The balloon is connected to a metal and plastic button that lies under the skin by means of a delicate silicone tube. This button that lies under the skin and fat fixed onto the abdomen muscle can be reached by a fine shot needle. Distilled water can, thus, be injected in order to tighten the stomach more or to empty the receiver so as to release the obstacle to the food passage.

This technique is prescribed to patients who are not very obese and who know they will not lose much weight (perhaps only 20% of the weight) and that do not like sweets or alcohol. This method's failure varies about 35%, as it depends a lot upon the patient and it also has postsurgical complications, such as:
Causing the esophagus dilatation by bringing about a serious difficulty for the esophageal emptying itself;
Slipping and causing total obstruction of the esophagus;
Suffering from erosion and getting perforated inside the stomach;
Suffering from infection due to contact with digestive liquid, skin, etc.

The other technique, the Intragastric Balloon (Bioenterics Intragastric Balloon—BIB®) is a silicone prosthesis with a spherical shape and a smooth surface that has a valve through which it is inflated inside the patient's stomach. It is introduced through the mouth and taken into the stomach, filled with about 700 ml of physiological saline solution. The presence of the balloon inside the stomach causes a stuffed feeling (full stomach), the so-called early satiety. Another more recent example is the Heliosphere Bag®, which has similar shape and material as the BIB® and it is also placed in the stomach; it is filled with gas, though. In all cases, the use of these balloons can only take place if all the recommended medical measures have already been applied and carried out, like diet and exercises, by the patients and the obtained results have been proven unsatisfactory.

One disadvantage is that the Intragastric Balloon constitutes a device for a temporary use (six months, at most), making it necessary to remove the balloon as it becomes corroded by stomach acids. It is, then, useful when intending to obtain but a modest reduction of patients weight, an act that reaches only a small number of obese people.

Therefore, according to the State of the Art, there are not materials, devices, techniques or procedures available are ideal in the treatment and the control of weight.

Innovations Introduced by this Patent Over the State of the Art.

The present patent is characterized by being a brand-new device which has been developed to control the esophageal flow; it is made of a biosynthetic material preferably based on but not limited to the natural latex extracted from the rubber tree Hevea brasiliensis, to be applied in the esophagus in order to control and limit the speed and amount of food intake and, thus, to provide weight loss. It preserves all digestive system functions, decreases the food intake due to the reduction of the esophagus lumen and in turn, decreasing the organ emptying, thus controlling the speed and amount of food intake.

### Manufacturing Process of the Esophageal Flow Controller.

The present ESOPHAGEAL FLOW CONTROLLER was developed by using natural latex extracted from the rubber tree Hevea brasiliensis as its raw material. From the natural latex, a final compound was prepared by adding chemical substances in order to give the product essential characteristics to the flow Controller. The characteristics are elasticity, softness, resistance, impermeability and hypoallergenic properties. After preparing the compost for manufacturing the Controller, filtering and dilution it in bi-distilled water stages must be done next. Every procedure must preferably be carried out in low temperature (below 20° C.) in order to prevent a pre-vulcanization of the liquid due to presence of heat.

For handling the latex, glass sticks were used for the mixing and a glass container used for storing it. Latex is a compound that becomes glue-like and gets vulcanized when in contact with the skin (due to the body temperature being ≈36° C.). For removing it, only pure water is used. Its removal from glass surfaces is eased due to the low friction that is present in the glass surface.

### 1—MANUFACTURING THE MOLDS

The esophagus is a very vascular, elastic, humid (it constantly receives the mouth secretions at short time intervals) organ surrounded by other organs that exert pressure on it, for instance, the lungs and the trachea.

Bearing these observations in mind, the mold was designed based on the anatomy and characteristics of the tissue wall of the esophageal organ. The purpose was to get a Controller that could be kept in a determined area by means of pressure and coefficient of friction against the esophagus wall, without needing any other mean of support. Thus, the Controller was developed and constituted of three distinct elements: the external module, the internal tube and the valve.

### A) EXTERNAL MODULE

The external module was manufactured from tubes, preferably of an inert polymer, with its dimension ranging between 5 and 10 cm long by 1.5 to 3.5 cm of external diameter with transversal grooves and there is a structure in the internal part of the external module for fitting in the valve during the setting up process, as it will be shown in the module manufacture issue.

### B) INTERNAL TUBE

The internal tube must be manufactured so that to have a smooth external surface and it must be compact and have 0.8 to 1.5 cm of external diameter and must be 5 to 10 cm long.

### C) VALVE

The valve mold must be preferably made of aluminum linked by an ordinary copper wire with the following measurements: the bigger pole being between 3 to 5 cm long, 1.5 to 3 mm of diameter, the smaller pole having 1.5 to 3 cm long and 1 to 2 mm of diameter. The distance between them is that of 3 to 7 mm.

### 2—MANUFACTURE OF THE ESOPHAGEAL FLOW CONTROLLER

After manufacturing the molds, they must be pre-washed with soap and water, dried with hot air and sterilized with a pressure boiler. During the manufacture process of the three distinct parts, the successive immersion bathing technique is used by introducing the molds inside the final latex compound gradually and uniformly, followed by heating them in a thermostatized oven.

The ready molds are then heated in the oven, at a temperature between 40° C. and 60° C., removed and soaked in the latex, left for at least 1 minute and then removed slowly and gradually from it. After that, they are placed in the oven (submitted to a vulcanization temperature heating which is never below 90° C.) at time intervals of three to ten minutes. They remain in the turned off oven for about 20 to 30 minutes. The bathing and heating steps must be repeated until the thicknesses of at least 2 mm for the external module and 1 mm for the internal module of the tube surface have been gotten. After the vulcanizing process, the mold is kept for at least 24 hours at room temperature for the manufacture process finalization. The valve must be manufactured in the same way as the other two parts. The next step is the removal of the mold Controller, which is made by using running water. In the process, the mold is placed in running water and the formed latex layer is removed little by little. After it is removed, the controller valve has the final dimensional characteristics of around 2.0 to 2.5 cm of length, a diameter of 0.3 to 0.7 cm and 0.3 to 0.6 cm of diameter in the solid part and 1 to 2 cm of diameter in the hollowed part.

After manufacturing the Controller by parts, the next step is setting it up. In the manufacture process, the successive immersion bathing technique is used, consisting of the introduction of the molds in the final latex compound at a perpendicular position, gradually and uniformly, followed by heating it up in a thermostatized oven.

The molds which were previously washed with soap and water, dried with hot air and sterilized with a pressure boiler must be heated in the oven with a temperature between 40° C. and 70° C., removed and soaked in latex, left for at least 1 minute, when, at this point, the beginning of the polymerization process that determines the product manufacture takes place. After that, they must be removed slowly and gradually, placed in the oven (submitted to a vulcanization temperature heating over 90° C.) in time intervals of three to ten minutes. Then, they are kept for 15 to 30 minutes in the turned off oven. The bathing and heating steps were repeated until reaching the thicknesses of at least 2 mm for the external module, 1 mm for the surface of the tube and of the radiographic indicator. After the vulcanizing period, the module was kept at room temperature for at least 24 hours in order to finalize the manufacture process. The valve was manufactured in the same way as the other three parts.

The removal of the mold pieces is made in running water, by removing the formed latex layer slowly so that the piece suffers no damage.

After manufacturing the pieces separately, the next step will be setting up the EFC. At this stage, the valve and radiographic indicator must be glued to the internal tube with latex. This procedure is carried out by inserting latex with a syringe and then placing the piece in the oven at over 90° C. until it is firmly stuck to the tube. A surgical wire (specific for medical application) of around 1 cm was also rolled around the internal tube from which its ending climbs up paralleled to the tube wall, being glued to it with the latex. It is advisable to leave at least 15 cm of the wire length outside the tube. This wire is purposed to fix the EFC externally and make its removal easy, by plucking it with tweezers and pulling it out of the body.

For a better understanding of the description, the EFC is shown as follows:

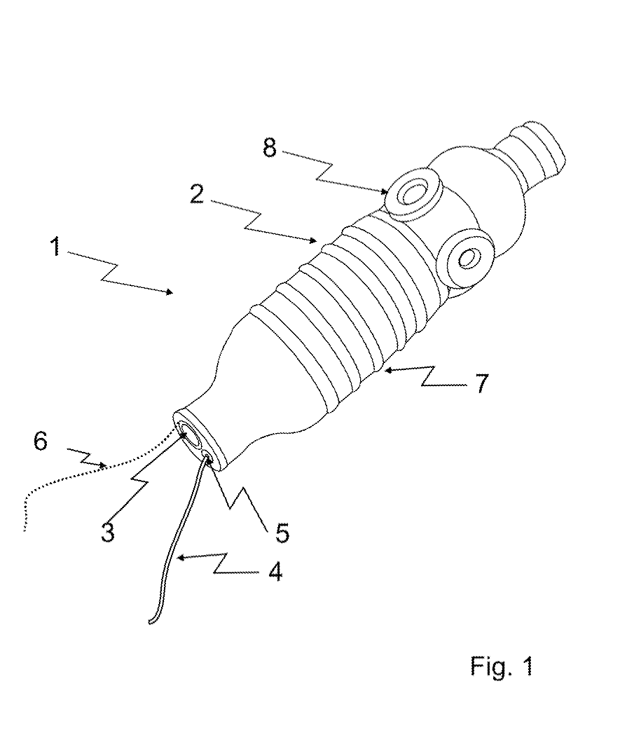

FIG. 1, from a perspective view, it is portrayed the whole EFC set (1) being formed by the external tube (2), internal tube (3), surgical wire (4), valve (5) and scalp (6), its transversal grooves (7) and suction cups (8).

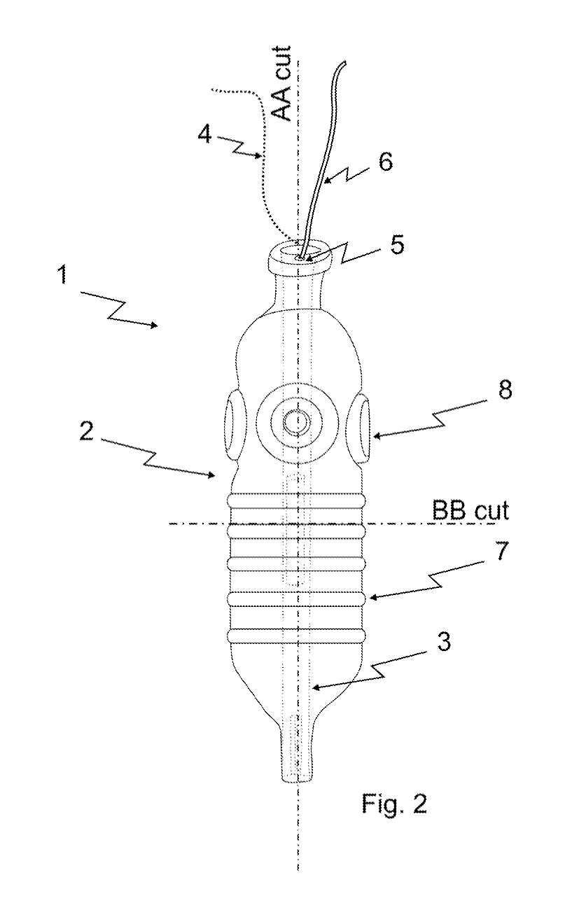

FIG. 2 shows in a frontal view, the whole EFC set (1), external tube (2), internal tube (3), surgical wire (4), valve (5) and scalp (6), its transversal grooves (7) and suction cups (8).

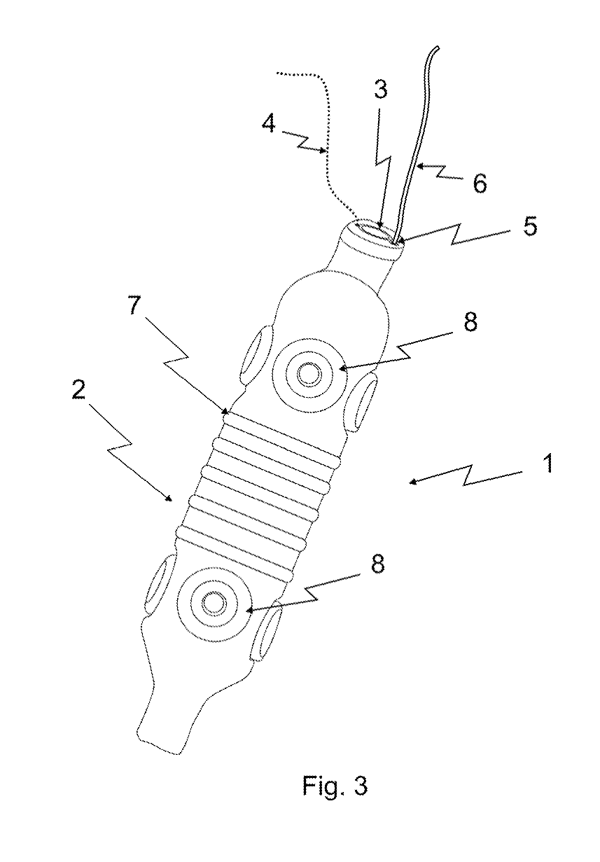

FIG. 3 shows a second constructive form for the external tube (2) where we can see a second group of suction cups (8) in the lower part, the upper side of internal tube (3), valve (5) placed in the module where the scalp (6) is fixed.

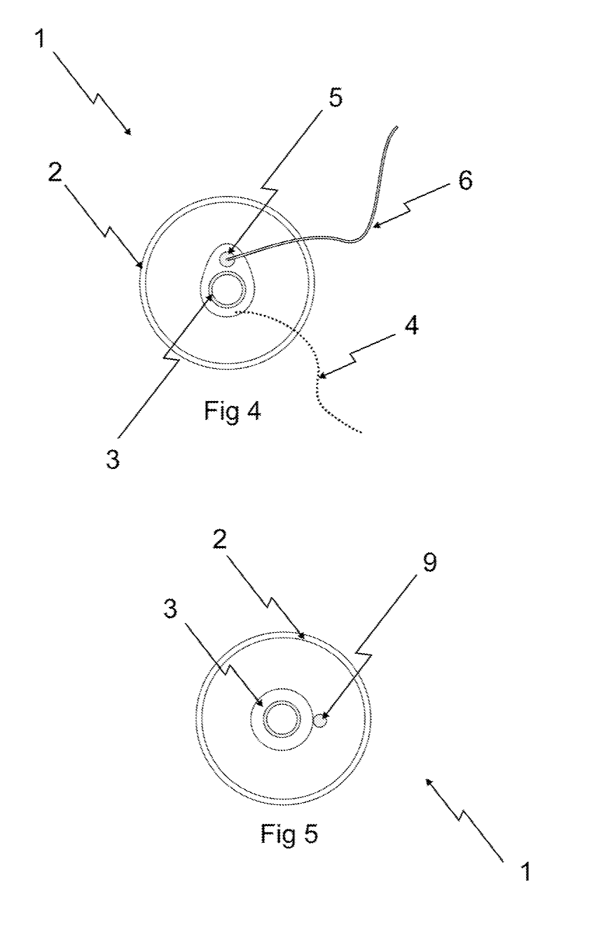

FIG. 4 shows an upper view of EFC set (1), where we can see external tube (2), internal tube (3), surgical wire (4), valve (5) and scalp (6).

FIG. 5 shows in a transverse BB cut, the wall of the internal tube (3), the wall of the external tube (2) and the radiographic indicator (9).

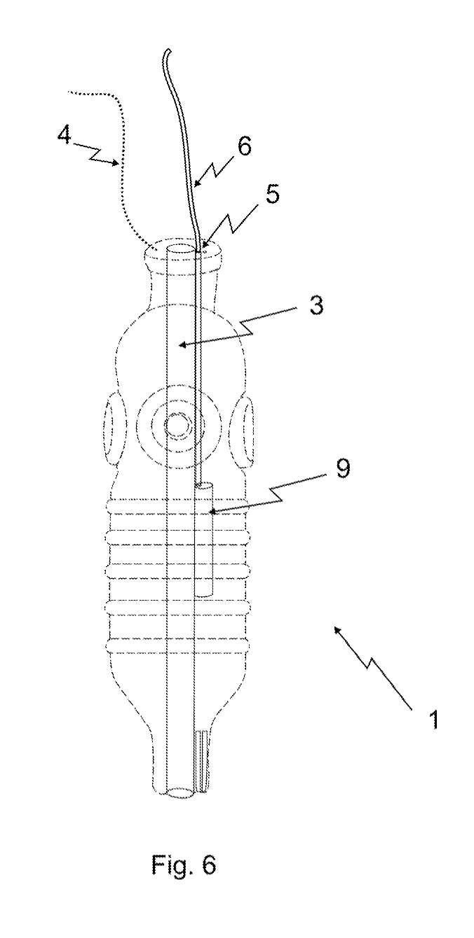

At last, FIG. 6 shows in a longitudinal AA cut, the surgical wire (4) fixed in the internal tube (3), valve (5) where the scalp (6) is placed and a radiographic indicator (9).

The macroscopic features of EFC are described in Table 1.

TABLE 1
Characteristics and dimensions of the EFC.

| Classification | Characteristics | Dimension |
|---|---|---|
| Shape | Cylindrical | 1.5 to 3.5 cm of external diameter |
| | | 1.5 to 2.5 mm of thickness |
| External | Waved with | 9 to 15 grooves of 2.0 mm to 5.0 mm |
| Surface | longitudinal grooves | of projection and 5.0 mm of space |
| | | between them. |
| Internal | Flat | 0.8 to 1.5 cm of hollowed diameter |
| Surface | | 1.5 to 2.5 mm of thickness |
| Color | Yellow | — |
| Length | — | 5.0 to 10 cm |

The new procedure called Esophageal Flow Controller module shown in the current work is a thoroughly original method, whose raw material is a natural component extracted from the Hevea brasiliensis.

The concept that generated this work and resulted in the present invention proposes the food intake reduction with the following characteristics:
1. The organ for it to be applied in is the esophagus—in its first third part, the organ has the function of just taking the food down, which would not cause the fundamental organ, which is the stomach, in the digestive process to change.
2. Mechanical reduction of the esophagus lumen, which will limit the amount of food to go from the mouth through to the stomach and limit the speed of solid food intake.
3. Application is through endoscopic via.
4. It does not perform any changes in the digestive system what will not cause dysfunctions or prevent the absorption of any nutrients.
5. It does not cause any esophageal damage whereas the gastric band does in 10% of the cases.

This new proposal method, based in the present EFC, is fit in the restrictive surgical techniques and it is empty when applied via an endoscope (non-surgical procedure—no need of hospitalization) into the first posterior third of the upper sphincter passage of the esophagus, at 3 cm to 8 cm deep, and it is then filled with gas.

1. “ESOPHAGEAL FLOW CONTROLLER”, to be used together with a technique for controlling and treating obesity, controlling the volume and speed of food intake, stimulating a higher individual's chewing rate decreasing the amount of food intake and controlling the esophageal flow, characterized by being adjustable to the esophagus lumen applied through endoscopic via into the first posterior third of the upper sphincter passage of the esophagus, at 3 cm to 8 cm deep afterwards inflated with gas; being provided with a flexible and inflatable external part made of a conductor duct with a fixed diameter, having a valve to let air in and having a flexible wire that enables its setting and removal and additionally having a radiographic indicator; the EFC being made of a biosynthetic material, preferably based on or made of, but not limited to, natural latex extracted from the rubber tree Hevea brasiliensis and is basically constituted by the whole EFC set (1), external tube (2), internal tube (3), surgical wire (4), valve (5), scalp (6), transversal grooves (7), suction cups (8) and a radiographic indicator (9).

2. “MANUFACTURING PROCESS OF THE ESOPHAGEAL FLOW CONTROLLER”, according to claim 1, characterized by the ESOPHAGEAL FLOW CONTROLLER being made preferably from the rubber tree Hevea brasiliensis as its raw material and by adding chemical substances to give the product adequate elasticity, softness, resistance, impermeability and bringing hypoallergenic properties procedures that must preferably be carried out in low temperature (below 20° C.); the obtained compost must be filtered and diluted in bi-distilled water stages.

3. “MANUFACTURING PROCESS OF THE MOLDS OF THE ESOPHAGEAL FLOW CONTROLLER”, according to claim 1, characterized by the external module being made from tubes, preferably of an inert polymer, with dimensions ranging between 5 cm and 10 cm long by 1.5 cm to 3.5 cm of external diameter with transversal grooves and having in the inner part a structure for fitting in the valve during the setting up process.

4. “MANUFACTURING PROCESS OF THE MOLDS OF THE ESOPHAGEAL FLOW CONTROLLER”, according to claim 1, characterized by the internal tube being manufactured so that it has a smooth external surface and is compact having 0.8 cm to 1.5 cm of external diameter and being 5 cm to 10 cm long.

5. “MANUFACTURING PROCESS OF THE MOLDS OF THE ESOPHAGEAL FLOW CONTROLLER”, according to claim 1, characterized by the valve mold being preferably made of aluminum linked by an ordinary copper wire with the following measurements: the bigger pole having 3 cm to 5 cm of length and 1.5 cm to 3 mm of diameter, the smaller pole having 1.5 cm to 3 cm long and 1 mm to 2 mm of diameter and a distance between them in the order of 3 to 7 mm.

6. “MANUFACTURING PROCESS OF THE ESOPHAGEAL FLOW CONTROLLER”, according to the claims above, characterized by the molds being pre-washed with soap and water, dried with the help of hot air afterwards and sterilized with a pressure boiler after manufactured.

7. “MANUFACTURING PROCESS OF THE ESOPHAGEAL FLOW CONTROLLER”, according to the claims above, characterized by having three distinct parts—tube (1), external tube (2), scalp (3) and a specific manufacturing process, using a successive immersion bathing technique by introducing the molds inside the final latex compound in a gradual and uniform manner, followed by heating the set in a thermostatized oven.

8. “MANUFACTURING PROCESS OF THE ESOPHAGEAL FLOW CONTROLLER”, according to claim 6, characterized by ready molds being heated in an oven at a temperature between 40° C. and 60° C., removed and soaked in the latex, left for at least 1 minute and then removed slowly and gradually from it; after what they are placed in the oven, submitted to a vulcanization temperature heating which is never below 90° C., at time intervals of three to ten minutes, afterwards remaining in the turned off oven for about 20 to 30 minutes; the bathing and heating steps having to be repeated until the thicknesses reach at least 2 mm for the wall on external module and 1 mm for the wall on internal module of the tube surface has been gotten; after the vulcanizing process the mold is kept for at least 24 hours at room temperature for the manufacturing process finalization.

9. “MANUFACTURING PROCESS OF THE ESOPHAGEAL FLOW CONTROLLER”, according to claim 6, characterized by the final step being the successive immersion bathing technique that is here used, consisting of the introduction of the molds in the final latex compound at a gradually and uniformly perpendicular position, followed by heating them in a thermostatized oven.

10. “MANUFACTURING PROCESS OF THE ESOPHAGEAL FLOW CONTROLLER”, according to claim 6, characterized by the molds, which were previously washed with soap and water, being dried with the help of hot air and afterwards sterilized with a pressure boiler and that they must be heated in the oven with a temperature between 40° C. and 70° C., removed and soaked in latex, left for at least 1 minute; after they must be removed slowly and gradually, placed in the oven (submitted to a vulcanization temperature heating over 90° C.) in time intervals of three to ten minutes. Then, molds are kept for 15 to 30 minutes in the turned off oven.

11. “MANUFACTURING PROCESS OF THE ESOPHAGEAL FLOW CONTROLLER”, according to claim 6, characterized by the bathing and heating steps that were repeatedly applied until reaching the thicknesses of at least 2 mm for the wall on the external module and 1 mm for the distance between the surface of the tube and of the radiographic indicator (9); after the vulcanizing period, the set of modules must be kept at room temperature for at least 24 hours in order to finalize the manufacturing process and allow for removal of the mold parts, what is done under running water by removing the formed latex layers slowly.

12. “MANUFACTURING PROCESS OF THE ESOPHAGEAL FLOW CONTROLLER”, according to claims above, characterized by the setting up of the ESOPHAGEAL FLOW CONTROLLER that begun with the fixing of the valve and of the radiographic indicator (9) to the internal tube with latex inserted by a syringe and then by placing the set in an oven at over 90° C. until said valve is firmly stuck to the tube; a surgical wire of around 1 cm is also rolled around the internal tube from which its ending climbs up in parallel to the tube's wall, being glued to it with the latex so that at least 15 cm of the wire length is left outside the tube.
